(19) United States
(12) Patent Application Publication
(10) Pub. No.: US 2017/0115264 A1
(43) Pub. Date: Apr. 27, 2017

(54) ANALYSIS APPARATUS FOR ANALYZING A GAS SAMPLE

(71) Applicant: SICK AG, Waldkirch/Breisgau (DE)

(72) Inventors: Christian KUHN, Ebringen (DE); Rolf SCHIFFLER, Kenzingen (DE); Daniel SCHIMANEK, Denzlingen (DE)

(21) Appl. No.: 15/334,391

(22) Filed: Oct. 26, 2016

(30) Foreign Application Priority Data
Oct. 26, 2015 (DE) 102015118208.4

(51) Int. Cl. G01N 33/00; G01N 21/61

(52) U.S. Cl. CPC G01N 33/0062; G01N 21/61; G01N 2021/1757

(57) ABSTRACT

The present invention relates to an analysis apparatus for analyzing a gas sample comprising a concentration measurement path receiving the gas sample; a light transmitter for transmitting light signals into the concentration measurement path; a detector for detecting light signals exiting the concentration measurement path; an evaluation unit which is adapted to determine the concentration of at least one substance present in the gas sample on the basis of the intensity of the detected light signals and on the basis of the length of the concentration measurement path; and a measurement device which is configured to determine the length of the time of flight path of the transmitted light signals optically using the light transmitter, with the evaluation unit being adapted to determine the length of the concentration measurement path on the basis of the determined length of the time of flight path.

The present invention relates to an analysis apparatus for analyzing a gas sample comprising a concentration measurement path receiving the gas sample; a light transmitter for transmitting light signals into the concentration measurement path; a detector for detecting light signals exiting the concentration measurement path; and an evaluation unit which is configured to determine the concentration of at least one substance present in the gas sample on the basis of the intensity of the detected light signals and on the basis of the length of the concentration measurement path.

Such analysis apparatus serve, for example, for the determination of the concentration of gases or of solid particles in industrial flue gas stacks, for monitoring flare gas or for monitoring vapor reclamation.

The light transmitter typically comprises a light source, for example a laser or a laser diode. Light in the sense of this application is understood as electromagnetic radiation whose wavelength is in the visible and/or non-visible range and which in particular also comprises infrared radiation or ultraviolet radiation. The determination of the concentration of at least one substance present in the gas sample is in particular also understood in the following as the determination of whether a specific substance is present in the gas sample or not, i.e. whether the substance is present in a concentration which is above the detection boundary of the analysis apparatus or which is above a predefined threshold.

The substance concentration is determined using the damping of the light signals transmitted by the light transmitter, optionally in a manner specific to the wavelength. It is thus possible, for example, to use or evaluate light of only one wavelength which is, for example, at a selected absorption peak of the gas component to be detected to determine the damping by this gas component. The light transmitter and the detector can be arranged at the same side or at different sides of the concentration measurement path. If the light transmitter and the detector are located at the same side of the concentration measurement path, a reflector is additionally provided which reflects the transmitted light signals after passing through the concentration measurement path in the direction of the detector so that the reflected light signals again pass through the concentration measurement path. In this case, it must be taken into account on the evaluation of the light signals that they have passed through the concentration measurement path twice so that the effective length of the concentration measurement path, i.e. the path distance effectively covered by the light signals, can also be covered by the term “length of the concentration measurement path”.

The gas sample can, for example, be a gas flow flowing in a flow passage or also an inactive gas volume received in a container. The concentration measurement path does not necessarily have to cover the total cross-section of the flow passage or of the container, but can rather e.g. also only comprise a part thereof, e.g. can extend from a wall up to a reflector within the flow passage or the container.

The length of the concentration measurement path has a substantial influence in the determination of the concentration of the at least one substance present in the gas sample since, as the length of the concentration measurement path increases, the damping of the light signals likewise increases with otherwise unchanging condition due to the laws of physics. The concentration is thus as a rule proportional to the quotient from the absorbed energy and the concentration measurement path. Changes of the length of the concentration measurement path during the operation of the analysis apparatus, for example due to thermal influences, can negatively influence the accuracy of the measurement. For this reason, U.S. Pat. No. 8,638,443 B2 proposes a monitoring of the length of the concentration measurement path by means of a turbulent flow sensor. The use of such a turbulent flow sensor is, however, complex and/or expensive in construction and can only be used with a limited concentration measurement path length.

It is the object of the invention to provide an analysis apparatus for analyzing a gas sample which is configured to consider the length of the concentration measurement path in a simple, reliable and inexpensive manner from a construction aspect.

The object is satisfied by an analysis apparatus having the features of claim 1.

An analysis apparatus in accordance with the invention comprises a concentration measurement path which receives the gas sample, a light transmitter for transmitting light signals into the concentration measurement path and a detector for detecting light signals exiting the concentration measurement path. An evaluation unit is adapted to determine the concentration of at least one substance present in the gas sample on the basis of the intensity of the detected light signals and on the basis of the length of the concentration measurement path. A measurement device is adapted to determine the length of the time of flight path of the transmitted light signals optically using the same light transmitter, with the evaluation unit being adapted to determine the length of the concentration measurement path on the basis of the determined length of the time of flight path. The used term “determination of the length of the concentration measurement path” is in this respect not to be understood such that such a determined length is actually also necessarily externally provided, but rather also such that this value is used directly, e.g. as part of an algorithm for determining the substance concentration.

The surprisingly simple idea underlies the invention of additionally using the light transmitter anyway present for the determination of the substance concentration for the determination of the length of the concentration measurement path. An additional signal source which only serves for the measurement of the concentration measurement path can be dispensed with. The maximum region within which a length determination of the concentration measurement path can be carried out in this manner as a rule also corresponds to the length region of the concentration measurement path within which a reliable determination of the substance concentration is possible in accordance with the specifications of the analysis apparatus. In other words, the possible length region of the concentration measurement path is generally not restricted by the measurement device, unlike, for example, the use of a separate turbulent flow sensor for determining the concentration measurement path.

The optical length measurement of the concentration measurement path is based on determining the length of the time of flight path covered by the transmitted light signals. One or more part sections of the time of flight path can also extend outside the gas sample for construction reasons, e.g. such a part section can extend between the light transmitter and/or the detector and a window which separates the light transmitter and/or the detector from the gas sample. Part sections of the time of flight path extending outside the gas sample can e.g. furthermore be caused by optionally present components such as windows and/or lenses or by regions flowed through by flushing gas. The length of the concentration measurement path therefore results from the optically determined length of the time of flight path covered by the light signals which is reduced by the length of optionally present part sections of the time of flight path extending outside the gas sample. The length of these part sections can be determined with knowledge of the construction circumstances.

The determined current length of the concentration measurement path can accordingly be directly considered in the determination of the substance concentration. A separate conversion or subsequent correction of the determined substance concentration is not necessary.

The measurement device is advantageously adapted to control the light transmitter directly or indirectly, with an indirect control being able to be implemented, for example, by a communication with a light transmitter control. The light signals can be transmitted in a continuous or pulsed form, with this in particular being able to be dependent on whether the light transmitter is operated for a concentration determination or a length determination. The invention is not restricted to analysis apparatus which are only operated at a specific wavelength, but rather also comprises spectroscopic analysis apparatus in which a tunable laser diode (TDLAS=“Tunable Diode Laser Absorption Spectroscopy”) is provided as a light transmitter or a wavelength-dispersing element connected upstream of the detector.

In accordance with an advantageous embodiment of the invention, an algorithm for calculating the substance concentration on the basis of the intensity of the detected light signals can be stored in the evaluation unit which uses the determined length of the concentration measurement path as the parameter. The determined length of the concentration measurement path is accordingly already considered in the determination of the substance concentration by the evaluation unit. A separate conversion or subsequent correction of the determined substance concentration is thus not necessary. The consideration of the current length of the concentration measurement path thus advantageously takes place “just-in-time”.

The measurement device and the evaluation unit can be configured as separate modules or can be combined in a common control unit. Alternatively, the evaluation unit can also take over the function of the measurement device so that a separate measurement device is dispensed with.

It is possible in a particularly advantageous manner generally to adapt the analysis apparatus for different lengths of the concentration measurement path, with the actual length of the concentration measurement path being determined once by means of a teaching process after the installation of the analysis apparatus and/or after a configuration change. The determined length of the concentration measurement path can subsequently be stored in the evaluation unit as a parameter for the concentration determination. The measurement device is therefore not only provided for an ongoing monitoring of the concentration measurement path length, bur can rather also be used for parameterization of the analysis apparatus.

The measurement device is advantageously adapted to determine the length of the time of flight path while also using the detector. Yet a further simplification of the analysis apparatus hereby results since not only the light transmitter, but also the detector can be used for both measurement procedures. It is alternatively possible also to use an additional dedicated detector for the determination of the time of flight path length. The analysis apparatus can be configured such that the light signals exiting the concentration measurement path can be received simultaneously by both detectors, e.g. with the aid of a beam splitter, or such that a selective reception by one of the two detectors is possible, e.g. by using adjustable beam-deflecting means such as mirrors or prisms.

In accordance with a further advantageous embodiment, the measurement device is adapted to determine the length of the time of flight path on the basis of the time of flight of light signals transmitted in the form of light pulses. The time of flight process is a generally known method for distance measurement which provides reliable measurement results over a large distance range. It is equally possible to determine the length of the time of flight path on the basis of a phase shift between the transmitted light signals and the detected light signals, with the measurement device in particular being adapted to modulate the light transmitter with a measurement frequency. When light signals having a modulated measurement frequency are used, the phase shift is evaluated with respect to the modulated measurement frequency so that an adaptation to the desired length range is possible by a suitable choice of the measurement frequency without the measurement result being falsified by ambiguities of the phase shift. The length of the time of flight path or of the concentration measurement path can furthermore also be determined geometrically, for example by means of optical triangulation. Since the length of the time of flight path covered by the light signals is as a rule determined by the named methods, this time of flight path—as mentioned above—optionally has to be corrected by the length of one or more part sections of the time of flight path which extend outside the gas sample to obtain the actual length of the concentration measurement path.

The evaluation unit is advantageously adapted to determine both the concentration of the at least one substance and the length of the concentration measurement path on the basis of the same light signal transmitted by the light transmitter. In other words, the length of the concentration measurement path and the substance concentration are determined simultaneously so that any length changes of the concentration measurement path can be considered without any time delay. Both the time of flight of a light pulse or the phase shift of a light signal and its intensity or intensity attenuation are therefore evaluated.

It is alternatively possible to provide an analysis apparatus which can be operated in two different modes of operation, with the one mode of operation enabling the concentration determination and the other mode of operation enabling the length measurement. This can in particular be provided when the length changes of the concentration measurement path to be expected only take place comparatively slowly.

In accordance with a further advantageous embodiment of the invention, the measurement device is adapted to determine the length of the time of flight path after the occurrence of a request event, with the request event comprising the elapse of a predefined time period and/or a temperature change and/or a user request influencing the length of the concentration measurement path. The length of the time of flight path or of the concentration measurement path can accordingly be determined in specific, preferably periodic, time intervals. A time of light path determination or a concentration measurement path determination can furthermore also be carried out after the determination of a temperature change in the region of the concentration measurement path, with a corresponding temperature sensor being able to be provided for a temperature monitoring of the gas sample, for example in a pipe wall of the flow passage or of the container. The named user request can be due, for example, to the carrying out of a teaching process in which the length of the concentration measurement path has to be determined for the first time after an installation of the analysis apparatus.

The invention also relates to a method of analyzing a gas sample which is located within a concentration measurement path, wherein the method comprises a transmission of light signals into the concentration measurement path by means of a light transmitter; a detection of light signals exiting the concentration measurement path; a determination of the concentration of at least one substance present in the gas sample on the basis of the intensity of the detected light signals; and a determination of the length of the concentration measurement path. The length of the concentration measurement path is determined optically using the light transmitter.

Advantageous embodiments of the method result from the description of the advantageous embodiments of the apparatus in accordance with the invention. The method in accordance with the invention can in particular be carried out using an analysis apparatus in accordance with at least one of the above-described embodiments.

The invention will be described in the following with reference to an embodiment and to the drawing. There is shown:

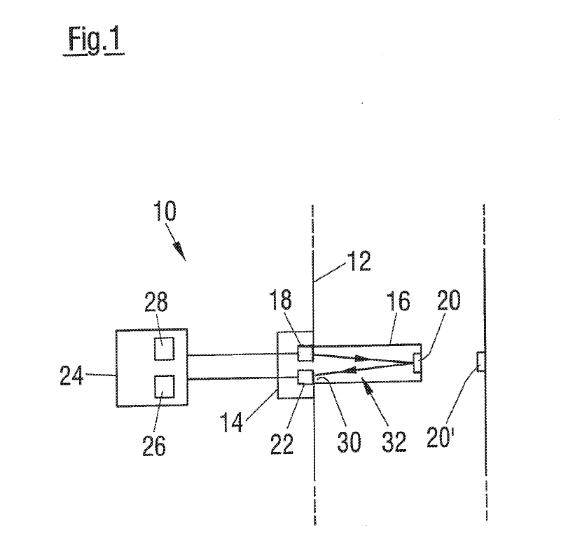

FIG. 1 a schematic representation of a sample space at which an analysis apparatus in accordance with the invention is arranged.

FIG. 1 shows in a simplified form an analysis apparatus 10 in accordance with the invention which is arranged at a sample space configured as a flow passage 12.

The analysis apparatus 10 comprises a measurement probe 14 arranged at the flow passage 12. The measurement probe 14 has a perforated measurement tube 16 which projects through an opening 30 provided in a wall of the flow passage 12 into the interior of the flow passage 12.

The measurement probe 14 comprises a light transmitter 18, for example a laser or a laser diode, which transmits light signals into the measurement tube 16. The light signals are reflected by a mirror 20 arranged at the end of the measurement tube 16 in the direction of a detector 22 arranged adjacent to the light transmitter 18.

The part of the time of flight path of the transmitted light signals which extends between the light transmitter 18, the mirror 20 and the detector 22 and which extends within the gas sample to be analyzed is called a concentration measurement path 32. The gas sample to be analyzed moves as a part of a gas flow flowing through the flow passage 12 through the apertures of the measurement tube 16 into the region of the concentration measurement path 32. Any part sections of the time of flight path extending outside the gas sample are not shown separately for reasons of clarity in FIG. 1.

In accordance with a modification, the measurement tube 16 and the mirror 20 can be dispensed with, with the light signals being reflected by a mirror 20′ (shown dashed) which is arranged at a wall of the flow passage 12 disposed opposite the opening 30.

In accordance with a further modification (not shown), the light transmitter and the detector can be arranged at oppositely disposed sides of the flow passage 12, with the mirror 20 or 20′ being able to be dispensed with and, optionally, also the measurement tube 16.

The measurement probe 14 is connected to a control unit 24 which comprises an evaluation unit 26 which is adapted to determine the concentration of at least one substance present in the gas sample on the basis of the intensity of the light signals detected by the detector 22 and on the basis of the length of the concentration measurement path 32.

The control unit 24 is adapted to determine the length of the concentration measurement path 32 on the basis of the time of flight path of the transmitted light signals, with the time of flight path being determined optically with the aid of a measurement device 28 provided in the control unit 24 while using the light transmitter 18 and the detector 22. In an embodiment which is not shown, the evaluation unit takes over the function of the measurement device so that the separate measurement device is dispensed with.

The length determination can take place in accordance with the time of flight method or on the basis of a phase shift between the transmitted light signals and the detected light signals. The length of the time of flight path hereby determined can optionally be reduced by the length of one or more part sections extending outside the gas sample to obtain the length of the concentration measurement path 32. These part sections are known from the geometry of the structure.

In the embodiment shown here, the detector 22 is used both for the measurement of the substance concentration and for the length measurement of the time of flight path or of the concentration measurement path 32. In accordance with a further modification, not shown, separate detectors can be provided for both measurements. It is thus possible, for example, to use a detector for the measurement of the substance concentration which is optimized with respect to the light sensitivity, whereas a detector is used for the length measurement of the time of flight path or of the concentration measurement path 32 which is optimized with respect to its response time.

The calculation of the substance concentration on the basis of the intensity of the detected light signals can take place in the evaluation unit 26 such that an algorithm is used for this purpose which uses the determined length of the concentration measurement path 22 as the parameter. For example, the evaluation unit 26 can evaluate the quotient form the absorbed energy and the length of the concentration measurement path 32, for this purpose.

The analysis apparatus 10 can determine both the substance concentration and the length of the concentration measurement path 32 on the basis of the same light signal transmitted by the light transmitter 18 by determining the time of flight or phase shift of this light signal while considering the described, optionally required corrections (in particular with respect to the optionally present difference between the time of flight path and the concentration measurement path). In this manner, the substance concentration and the length of the time of flight path or of the concentration measurement path are determined simultaneously such that no time delay occurs on the consideration of length changes. The method steps of determining the concentration of at least one substance present in the gas sample and the method step of determining the length of the time of flight path or of the concentration measurement path thus take place simultaneously or synchronously.

Alternatively, the analysis apparatus 10 can be provided such that it can be operated in two different modes of operation, with the measurement of the substance concentration taking place in the one mode of operation and the length measurement taking place in the other mode of operation. The two above-named method steps are carried out after one another in time in this respect.

### REFERENCE NUMERAL LIST

10 analysis apparatus
12 flow passage
14 measurement probe
16 measurement tube
18 light transmitter
20, 20′ mirror
22 detector
24 control unit
26 evaluation unit
28 measurement device
30 opening
32 concentration measurement path

1. An analysis apparatus for analyzing a gas sample, comprising
a concentration measurement path having a length and receiving the gas sample;
a light transmitter for transmitting light signals into the concentration measurement path;
a detector for detecting light signals exiting the concentration measurement path;
an evaluation unit adapted to determine a concentration of at least one substance present in the gas sample on the basis of an intensity of the detected light signals and on the basis of the length of the concentration measurement path; and
a measurement device adapted to determine a length of a time of flight path of the transmitted and detected light signals optically using the same light transmitter, with the evaluation unit further being adapted to determine the length of the concentration measurement path on the basis of the determined length of the time of flight path.

2. The analysis apparatus in accordance with claim 1,
wherein an algorithm for calculating the concentration of the at least one substance on the basis of the intensity of the detected light signals is stored in the evaluation unit and the algorithm uses the length of the concentration measurement path determined on the basis of the time of flight path as a parameter.

3. The analysis apparatus in accordance with claim 1,
wherein the measurement device is adapted to determine the length of the time of flight path using the detector.

4. The analysis apparatus in accordance with claim 1,
wherein the measurement device is adapted to determine the length of the time of flight path on the basis of the time of flight of the transmitted light signals.

5. The analysis apparatus in accordance with claim 1,
wherein the measurement device is adapted to determine the length of the time of flight path on the basis of a phase shift between the transmitted light signals and the detected light signals.

6. The analysis apparatus in accordance with claim 5,
wherein the measurement device is adapted to modulate the light transmitter with a measurement frequency.

7. The analysis apparatus in accordance with claim 1,
wherein the evaluation unit is adapted to determine both the concentration of the at least one substance and the length of the concentration measurement path on the basis of the same light signal transmitted by the light transmitter.

8. The analysis apparatus in accordance with claim 1,
wherein the measurement device is adapted to determine the length of the time of flight path after the occurrence of a request event,
with the request event comprising at least one of an elapse of a predefined time period, a temperature change and a user request influencing the length of the concentration measurement path.

9. A method of analyzing a gas sample which is located within a concentration measurement path having a length, comprising the steps:
transmitting light signals into the concentration measurement path by means of a light transmitter;
detecting light signals exiting the concentration measurement path; and
determining a concentration of at least one substance present in the gas sample on the basis of an intensity of the detected light signals and on the basis of the length of the concentration measurement path;
with the length of the concentration measurement path being determined optically using the light transmitter.

10. The method in accordance with claim 9,
further comprising the step of:
determining the concentration of the at least one substance on the basis of the determined length of the concentration measurement path.

11. The method in accordance with claim 10, wherein the step of determining the concentration of the at least one substance on the basis of the determined length of the concentration measurement path is carried out using an algorithm for calculating the substance concentration on the basis of the intensity of the detected light signals which uses the determined length of the concentration measurement path as a parameter.

12. The method in accordance with claim 9,
wherein both the concentration of the at least one substance and the length of the concentration measurement path are determined on the basis of the same light signal transmitted by the light transmitter.
